(19) United States
(12) Patent Application Publication
(10) Pub. No.: US 2025/0295389 A1
(43) Pub. Date: Sep. 25, 2025

(54) ULTRASOUND DIAGNOSTIC APPARATUS

(71) Applicant: FUJIFILM Corporation, Tokyo (JP)

(72) Inventor: Akinori SATO, Tokyo (JP)

(73) Assignee: FUJIFILM Corporation, Tokyo (JP)

(21) Appl. No.: 19/086,098

(22) Filed: Mar. 20, 2025

(30) Foreign Application Priority Data
Mar. 22, 2024 (JP) 2024-045892

(51) Int. Cl. A61B 8/00

(52) U.S. Cl. CPC A61B 8/4427; A61B 8/4405

(57) ABSTRACT

Provided is an ultrasound diagnostic apparatus. A monitor part is openable and closable with respect to an apparatus main body and is revolvable around a revolution axis. The monitor part is biased toward a closing position in the vicinity of the closing position. The revolution axis is inclined rearward with respect to a direction perpendicular to the operation panel provided on an upper surface of the apparatus main body. In a case where the monitor part that is at the closing position revolves around the revolution axis inclined rearward, a locus of a point on the monitor part interferes with the apparatus main body. Therefore, revolution of the monitor part that is at the closing position is suppressed since the monitor part and the apparatus main body interfere with each other due to the revolution.

### CROSS-REFERENCE TO RELATED APPLICATION

This application claims the priority benefit of Japan application serial no. 2024-045892, filed on Mar. 22, 2024. The entirety of the above-mentioned patent application is hereby incorporated by reference herein and made a part of this specification.

### BACKGROUND OF THE INVENTION

### 1. Field of the Invention

The present invention relates to an ultrasound diagnostic apparatus, and particularly to an ultrasound diagnostic apparatus in which an apparatus main body and a monitor part are closed in a case where the ultrasound diagnostic apparatus is not to be used.

### 2. Description of the Related Art

An ultrasound diagnostic apparatus including an apparatus main body that includes an operation panel provided with a keyboard or a pointing device and a monitor part that is supported to be rotationally movable with respect to the apparatus main body is known. In a case where the ultrasound diagnostic apparatus is not to be used, the monitor part is laid over the apparatus main body to be moved to a closing position and in a case where the ultrasound diagnostic apparatus is to be used, the monitor part is rotated and raised to be moved to an opening position.

Disclosed in JP 2008-253592A is an ultrasound diagnostic apparatus (10) including a main body (12) that includes a display unit (15) and an operation panel (14). In a case where the ultrasound diagnostic apparatus (10) is not to be used, the main body (12) and the operation panel (14) are closed such that the display unit (15) is positioned on an inner side. A guide plate (108) and a follower protrusion (106) provided at a slide rod (104) are engaged with each other so that the main body (12) and the operation panel (14) are kept closed.

Disclosed in JP 2010-240072A is an ultrasound diagnostic apparatus (10) including a main body unit (12), a lower panel (18) that includes a display surface (40), an upper panel (20) that includes a main display surface (44), and a flat plate-shaped auxiliary member (22). In a case where the ultrasound diagnostic apparatus (10) is not to be used, the lower panel (18), the upper panel (20), and the auxiliary member (22) are folded and laid over the main body unit (12).

It should be noted that the above reference numerals in parentheses are reference numerals used in JP2008-253592A and JP2010-240072A and are not related to reference numerals used in the description of the embodiment of the present application.

### SUMMARY OF THE INVENTION

In a case where an ultrasound diagnostic apparatus including a monitor part that is openable and closable with respect to an apparatus main body and in which the monitor part is revolvable is transported, the closed monitor part may revolve. The present invention suppresses revolution of a closed monitor part.

An ultrasound diagnostic apparatus according to an aspect of the present invention includes an apparatus main body that includes an operation panel, a monitor part that is movable between a closing position at which the monitor part is laid over the operation panel and an opening position at which the monitor part is erected on a rear edge portion of the apparatus main body, and a hinge member that is supported by the apparatus main body at the rear edge portion of the apparatus main body and that supports the monitor part. The hinge member is supported by the apparatus main body to be rotationally movable around a revolution axis inclined rearward with respect to a direction perpendicular to the operation panel, the monitor part revolving as the hinge member rotationally moves around the revolution axis. In addition, the hinge member supports the monitor part to be rotationally movable around an opening and closing axis orthogonal to a direction in which the revolution axis extends, the monitor part being opened and closed between the closing position and the opening position as the monitor part rotationally moves around the opening and closing axis. Furthermore, the hinge member includes a biasing mechanism that biases, toward the closing position, the monitor part positioned in a vicinity of the closing position.

Since the monitor part is rotatable around the revolution axis inclined rearward with respect to the operation panel, in a case where the monitor part that is at the closing position revolves, the monitor part and the apparatus main body interfere with each other. Therefore, revolution of the monitor part is suppressed. In addition, since the monitor part is biased toward the closing position by the biasing mechanism, a rise of the monitor part attributable to revolution of the monitor part that is at the closing position is suppressed. As a result, the biasing mechanism biasing the monitor part contributes to suppression of revolution of the monitor part.

Furthermore, in a case where the ultrasound diagnostic apparatus is installed at a predetermined position, a front side of the operation panel may be lowered and the revolution axis may extend along a vertical direction.

Since revolution of the monitor part that is at the closing position is suppressed, movement of the monitor part can be suppressed in the case of transportation of the ultrasound diagnostic apparatus or the like.

### BRIEF DESCRIPTION OF THE DRAWINGS

FIG. 1 is a front perspective view of an ultrasound diagnostic apparatus of the present embodiment that is in a state of being mounted on a cart.

FIG. 2 is a rear perspective view of the ultrasound diagnostic apparatus according to the present embodiment that is in a state of being mounted on the cart.

FIG. 3 is a perspective view showing the ultrasound diagnostic apparatus of the present embodiment and shows a state where a monitor part is open.

FIG. 4 is a side view showing the ultrasound diagnostic apparatus according to the present embodiment and shows a state where the monitor part is open.

FIG. 5 is a rear view showing the ultrasound diagnostic apparatus of the present embodiment and shows a state where the monitor part is closed.

FIG. 6 is a front view showing a hinge member.

FIG. 7 is a side view showing the hinge member.

FIG. 8 is a plan view showing the hinge member.

FIGS. 9A and 9B are explanatory views of a biasing mechanism that biases the monitor part toward a closing position.

FIGS. 10A and 10B are explanatory views about suppression of revolution of the monitor part that is at the closing position.

### DESCRIPTION OF THE PREFERRED EMBODIMENTS

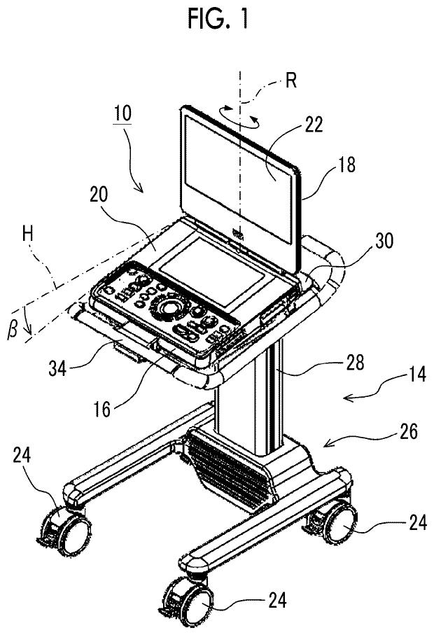

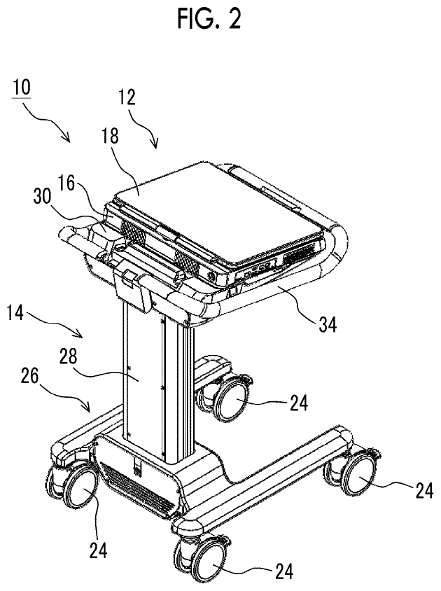

Hereinafter, an embodiment of the present invention will be described with reference to the drawings. FIGS. 1 and 2 are views showing an ultrasound diagnostic apparatus 10 in a state of being installed on a cart 14. FIG. 1 is a perspective view showing a front side, and FIG. 2 is a perspective view showing a rear side. The ultrasound diagnostic apparatus 10 includes an apparatus main body 16 having a flat rectangular parallelepiped-like shape and a monitor part 18 having a flat plate-like shape that is supported at a rear edge portion of the apparatus main body 16. As shown in FIG. 2, in a case where the monitor part 18 is laid over the apparatus main body 16, the shape of the ultrasound diagnostic apparatus 10 becomes a substantially rectangular parallelepiped-like shape as a whole. The ultrasound diagnostic apparatus 10 is used after the monitor part 18 is rotationally moved in such a state to be upright as shown in FIG. 1. The apparatus main body 16 accommodates a circuit that processes transmission and reception signals of ultrasonic waves, a circuit that processes an image to be displayed, a power supply circuit, a battery, a storage device, and the like. An operation panel 20 including a pointing device such as a trackball and a trackpad and buttons for operation is provided at an upper surface of the apparatus main body 16. A display 22 is disposed at the monitor part 18.

The cart 14 includes a leg portion 26 including four casters 24, a columnar portion 28 erected on the leg portion 26, and a placement table 30 fixed to an upper end of the columnar portion 28. The ultrasound diagnostic apparatus 10 is placed on the placement table 30. Around the placement table 30, a cart handle 34 is provided over the substantially entire circumference of the placement table 30.

The ultrasound diagnostic apparatus 10 can be transported in a state of being installed on the cart 14 and can be transported alone after being removed from the cart 14.

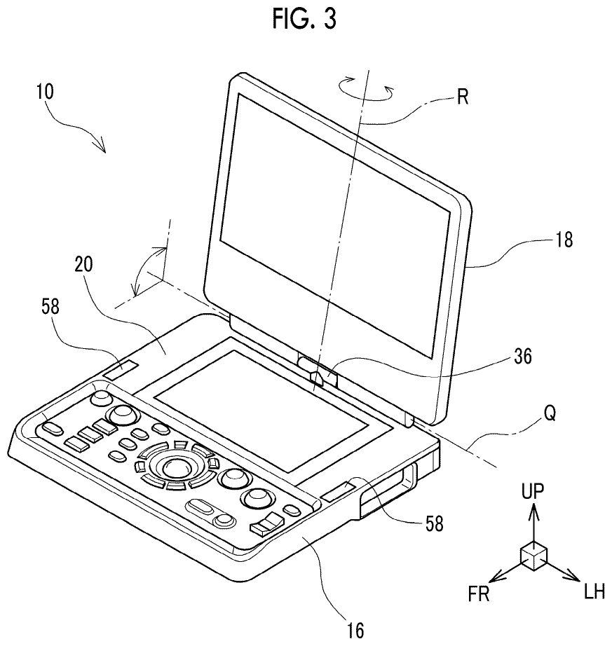

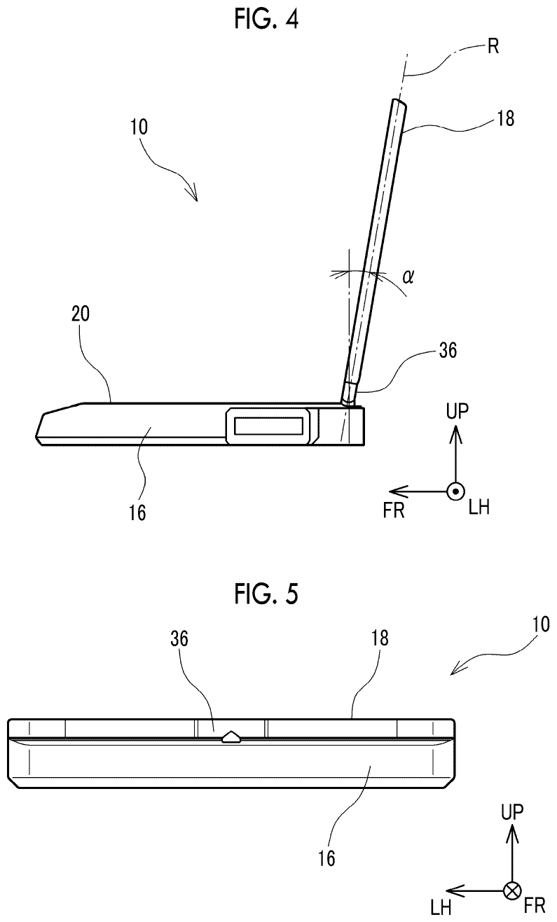

FIGS. 3 to 5 are views showing the ultrasound diagnostic apparatus 10 alone. In FIGS. 3 to 5 and subsequent drawings, a direction along an arrow FR is a forward direction, a direction along an arrow UP is an upward direction, and a direction along an arrow LH is a leftward direction. FIG. 3 is a perspective view showing a state where the monitor part 18 is open, and the monitor part 18 is erected on the rear edge portion of the apparatus main body 16. FIG. 4 is a side view showing a state where the monitor part 18 is open. FIG. 5 is a rear view showing a state where the monitor part 18 is closed.

In a case where the ultrasound diagnostic apparatus 10 is placed on a horizontal surface, the operation panel 20 is also disposed horizontally. The monitor part 18 is supported by a hinge member 36 at the rear edge portion of the apparatus main body 16. The hinge member 36 supports the monitor part 18 to be rotationally movable around a revolution axis R and supports the monitor part 18 to be rotationally movable around an opening and closing axis Q as well, the opening and closing axis Q being orthogonal to a direction in which the revolution axis R extends. In the ultrasound diagnostic apparatus 10, the revolution axis R and the opening and closing axis Q are orthogonal to each other. Hereinafter, rotational movement of the monitor part 18 around the revolution axis R will be referred to as revolution, and rotational movement of the monitor part 18 around the opening and closing axis Q will be referred to as being opened and closed.

The revolution axis R is fixed with respect to the apparatus main body 16 and is inclined rearward with respect to a vertical direction in FIGS. 3 to 5, that is, a direction perpendicular to a surface of the operation panel 20. A rearward inclination angle α may be 5° to 15°, and particularly may be 10°. In addition, the rearward inclination angle α may be determined such that the revolution axis R extends in a vertical direction in a case where the ultrasound diagnostic apparatus 10 is placed on the cart 14. As shown in FIG. 1, the ultrasound diagnostic apparatus 10 may be placed on the cart 14 to be inclined with respect to a horizontal line such that a front edge of the operation panel 20, that is, an edge of the operation panel 20 that is on a side ahead of an operator is lowered. A one-dot chain line H shown in FIG. 1 represents the horizontal line, and the operation panel 20 is inclined at an angle β. In a case where the rearward inclination angle α is made equal to the angle β at which the operation panel 20 is inclined, the revolution axis R extends in the vertical direction. The opening and closing axis Q is revolved together with the monitor part 18 and in a case where the monitor part 18 faces a front side, the opening and closing axis Q is disposed horizontally, that is, disposed parallel to the surface of the operation panel 20.

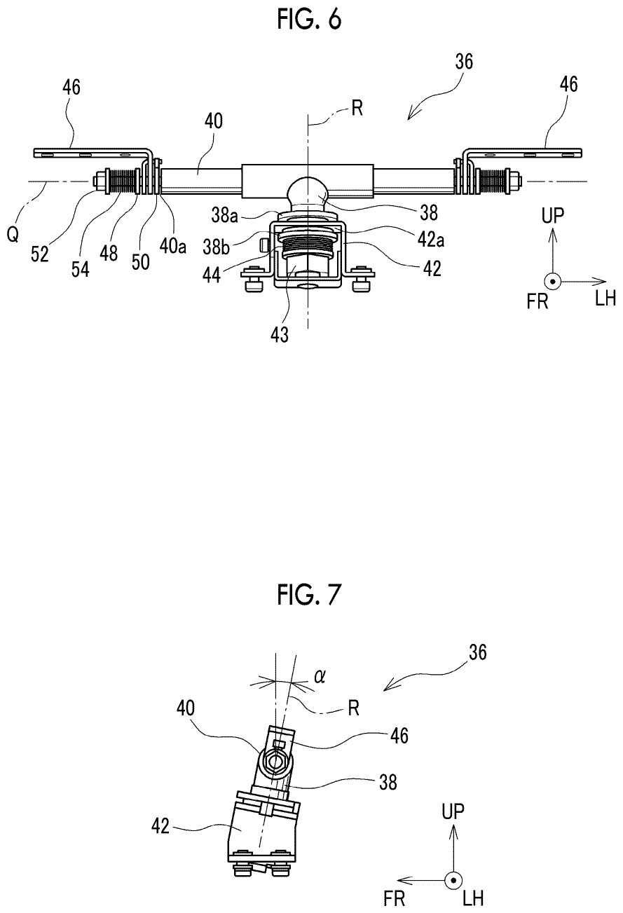

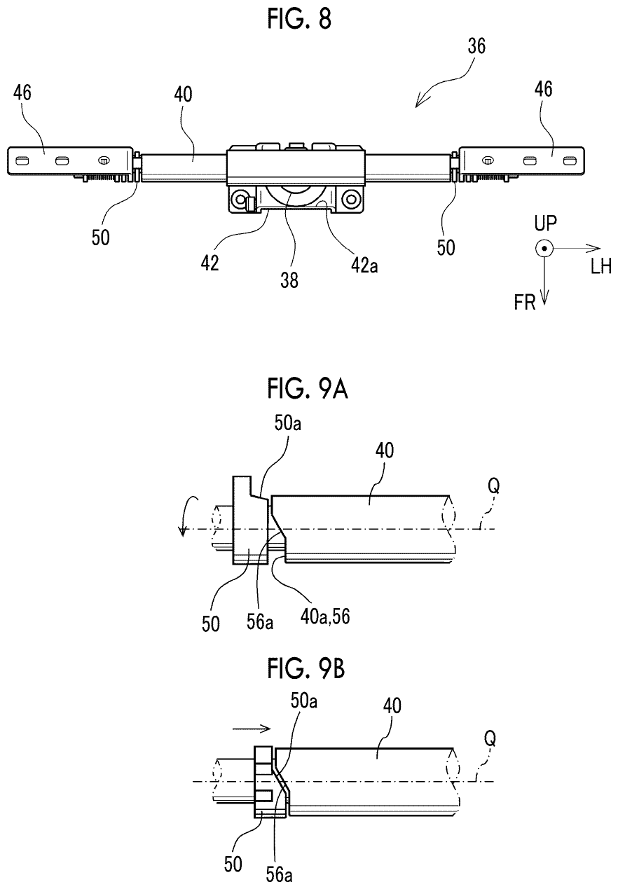

FIGS. 6 to 8 are views showing the hinge member 36, where FIG. 6 is a front view, FIG. 7 is a side view, and FIG. 8 is a plan view. The hinge member 36 includes a stem 38 and a hinge shaft 40 disposed along a direction orthogonal to the stem 38. The stem 38 and the hinge shaft 40 are integrated with each other and may be disposed in a substantially T-like shape, and a central portion of the hinge shaft 40 is connected to an upper end of the stem 38. The stem 38 is supported to be rotationally movable around the revolution axis R by a main body bracket 42 fixed to the apparatus main body 16. Therefore, a center line of the stem 38 coincides with the revolution axis R. The monitor part 18 revolves around the stem 38. The stem 38 is supported by the main body bracket 42 with a connection plate portion 42a of the main body bracket 42 sandwiched between a flange 38a and a washer 38b of the stem 38. In a case where a nut 43 that is screwed onto a lower end portion of the stem 38 by being screwed is tightened, a biasing force acts on the washer 38b via a plurality of laminated disc springs 44 and the connection plate portion 42a is sandwiched due to the biasing force. The position of the monitor part 18 in a revolution direction is maintained due to friction between the flange 38a, the washer 38b, and the connection plate portion 42a.

The hinge shaft 40 supports L-shaped monitor brackets 46, which are fixedly connected to the monitor part 18, at right and left end portions to be rotationally movable around the opening and closing axis Q. A portion of each monitor bracket 46 that extends in a lateral direction is fastened to a lower edge portion of the monitor part 18 by means of a fastening element such as a bolt. End portions of the hinge shaft 40 have a small diameter so that shoulder portions 40a are formed and cam follower washers 50 and the monitor brackets 46 are sandwiched by the shoulder portions 40a and washers 48. The cam follower washers 50 are integrated with the monitor brackets 46 in relation to rotation around the opening and closing axis Q and the cam follower washers 50 rotationally move together with the monitor brackets 46 rotationally moving as the monitor part 18 is opened and closed. In a case where nuts 52 that are screwed onto the end portions of the hinge shaft 40 by being screwed are tightened, biasing forces act on the washers 48 via a plurality of laminated disc springs 54. A biasing force from a left side acts on the monitor bracket 46 on the left side and a biasing force from a right side acts on the monitor bracket 46 on the right side, so that a pair of right and left monitor brackets 46 is sandwiched by the biasing forces. A resistance against an opening and closing motion of the monitor part 18 is applied due to friction acting between the monitor brackets 46 and the disc springs 54.

FIGS. 9A and 9B are enlarged views showing a cam 56 formed at the shoulder portion 40a of the hinge shaft 40 and the cam follower washer 50. FIG. 9A shows a state where the monitor part 18 is at an opening position and FIG. 9B shows a state where the monitor part 18 is at a closing position. The cam 56 serving as an edge surface cam is formed at the shoulder portion 40a of the hinge shaft, and as shown in the drawings, the cam 56 includes an inclined surface 56a. An inclined surface 50a is also formed on a surface of the cam follower washer 50 that faces the cam 56.

The cam follower washer 50 rotationally moves together with the monitor bracket 46. However, the cam follower washer 50 is movable independently of the monitor bracket 46 in a direction along the opening and closing axis Q. The cam follower washer 50 is biased in a direction toward the shoulder portion 40a, that is, in a rightward direction in FIGS. 9A and 9B, and is always in contact with the shoulder portion 40a. Such a biasing force may be applied by a spring provided between the cam follower washer 50 and the monitor bracket 46 (for example, a disc spring).

In a case where the monitor part 18 is at the opening position, the inclined surface 56a of the cam 56 and the inclined surface 50a of the cam follower washer 50 do not face each other as shown in FIG. 9A. In a case where the monitor part 18 is at the closing position, the inclined surfaces 50a and 56a face each other as shown in FIG. 9B.

In a case where the inclined surface 50a of the cam follower washer comes into contact with a portion of the inclined surface 56a of the cam while the monitor part 18 is rotationally moving from the opening position to the closing position, the cam follower washer 50 is prompted to rotationally move toward the closing position by a biasing force acting on the cam follower washer 50. In a case where the monitor part 18 is brought close to the closing position due to the biasing force acting on the cam follower washer 50, a biasing force toward the closing position acts on the monitor part 18. The cam 56, the cam follower washer 50, and a spring that biases the cam follower washer 50 toward the cam 56 constitute a biasing mechanism that biases the monitor part 18 toward the closing position. A biasing force toward the closing position may act in a range within 5° to 15° from the closing position, and preferably may act in a range within 10° from the closing position. Such a biasing force also acts as a force that holds the monitor part 18 at the closing position. In addition, in a case where the monitor part 18 is far from the closing position, the biasing force may not act.

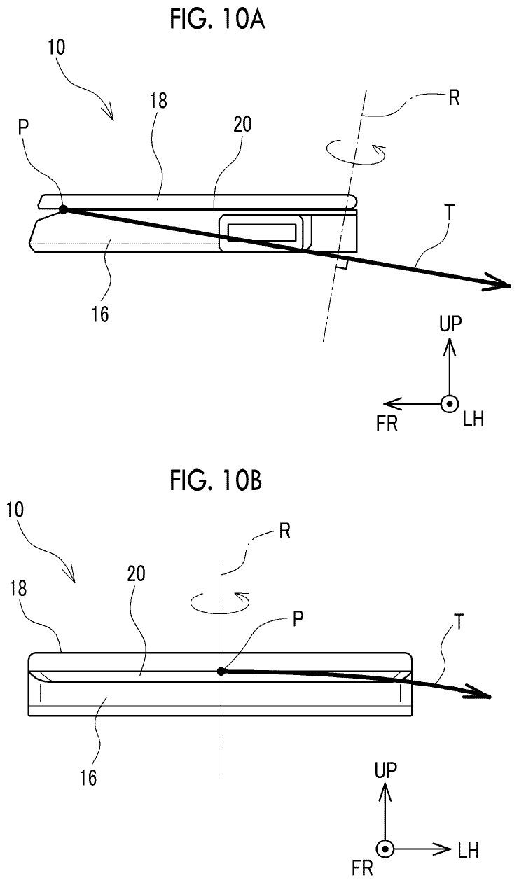

FIGS. 10A and 10B are explanatory views about suppression of revolution of the monitor part 18 that is at the closing position. FIG. 10A is a side view, and FIG. 10B is a front view. In a case where the monitor part 18 revolves around the revolution axis R, any point P on the monitor part 18 draws an arc-shaped locus T centered on the revolution axis R within a plane orthogonal to the revolution axis R. Since the revolution axis R is inclined rearward, the locus T of the point P interferes with the apparatus main body 16. Therefore, the monitor part 18 that is at the closing position interferes with the apparatus main body 16 in a case where the monitor part 18 is about to revolve, so that revolution thereof is suppressed. In addition, a biasing force that moves the monitor part 18 to the closing position suppresses a rise of the monitor part 18 that occurs in a case where the monitor part 18 is about to revolve, which contributes to suppression of revolution of the monitor part 18.

Revolution of the monitor part 18 that is at the closing position, which occurs due to the monitor part 18 vibrating and shaking at the time of movement in a case where the ultrasound diagnostic apparatus 10 is moved after being placed on the cart 14, a case where the ultrasound diagnostic apparatus 10 is transported alone, or the like, can be suppressed.

For suppression of revolution of the monitor part 18 that is at the closing position, friction pads 58 (refer to FIG. 3) may be disposed in the vicinities of right and left edges of the operation panel 20. The friction pads 58 may be made of a material having a high friction coefficient, such as rubber. Furthermore, the friction pads may be disposed in the vicinities of right and left edges of a surface of the monitor part 18 that faces the operation panel 20, instead of being provided at the operation panel 20 or in addition to being provided at the operation panel 20.

What is claimed is:

1. An ultrasound diagnostic apparatus comprising:
an apparatus main body that includes an operation panel;
a monitor part that is movable between a closing position at which the monitor part is laid over the operation panel and an opening position at which the monitor part is erected on a rear edge portion of the apparatus main body; and
a hinge member that is supported by the apparatus main body at the rear edge portion of the apparatus main body and that supports the monitor part,
wherein the hinge member is supported by the apparatus main body to be rotationally movable around a revolution axis inclined rearward with respect to a direction perpendicular to the operation panel, the monitor part revolving as the hinge member rotationally moves around the revolution axis,
the hinge member supports the monitor part to be rotationally movable around an opening and closing axis orthogonal to a direction in which the revolution axis extends, the monitor part being opened and closed between the closing position and the opening position as the monitor part rotationally moves around the opening and closing axis, and
the hinge member includes a biasing mechanism that biases, toward the closing position, the monitor part positioned in a vicinity of the closing position.

2. The ultrasound diagnostic apparatus according to claim 1, wherein
in a case where the ultrasound diagnostic apparatus is installed at a predetermined position, a front side of the operation panel is lowered and the revolution axis extends along a vertical direction.
